(19) United States
(12) Patent Application Publication
(10) Pub. No.: US 2024/0315187 A1
(43) Pub. Date: Sep. 26, 2024

(54) MICROPROPAGATION MEDIUM

(71) Applicant: PROFILE PRODUCTS LLC, Buffalo Grove, IL (US)

(72) Inventors: Edwin Robert VALSTAR, Den Haag (NL); Sjoerd A. YPMA, Kwintsheul (NL); Jurriën COLLET-MARTOPAWIRO, Haarlem (NL)

(73) Assignee: PROFILE PRODUCTS LLC, Buffalo Grove, IL (US)

(21) Appl. No.: 18/615,464

(22) Filed: Mar. 25, 2024

Related U.S. Application Data
(60) Provisional application No. 63/492,103, filed on Mar. 24, 2023.

(51) Int. Cl. A01H 4/00

(52) U.S. Cl. CPC A01H 4/002; A01H 4/001; A01H 4/005

(57) ABSTRACT

A propagation plug includes an irradiated, hydrophilic substrate body comprising a growing medium having bound organic fibers, the substrate body being rewettable and having a moisture content of less than about 20 wt. %, based on the total weight of the body, as measured according to ASTM test method no. D2216 and being sterile as evidenced by having an aerobic bacterial count of <10 cfu/g as measured by ISO test method no. 4833-1.

### CROSS-REFERENCE TO RELATED APPLICATIONS

This application claims the benefit of U.S. provisional application Ser. No. 63/492,103 filed Mar. 24, 2023, the disclosure of which is hereby incorporated in its entirety by reference herein.

### TECHNICAL FIELD

The present disclosure relates to a tissue culture propagation medium, in at least one embodiment a medium for in vitro to ex vitro tissue culture propagation, and methods of making and using the same.

### BACKGROUND

Plant propagation relates to a process by which new plants grow from a variety of sources such as seeds, cuttings, and other plant parts. To support the initial germination and growth, various media have been developed. Micropropagation or tissue culture is a type of plant propagation.

### SUMMARY

In one embodiment, a micropropagation medium is disclosed. The medium is a rewettable, irradiated fibrous material having a moisture content of less than about 20% before irradiation. The micropropagation medium may be structured as a self-supporting plug. The micropropagation medium may further include a tray having a plurality of cells, at least some of the cells housing the micropropagation medium.

In an embodiment, a propagation plug is disclosed. The propagation plug includes an irradiated, hydrophilic substrate body comprising a growing medium having bound organic fibers, the substrate body being rewettable and having a moisture content of less than about 20 wt. %, based on the total weight of the body, as measured according to ASTM test method no. D2216 and being sterile as evidenced by having an aerobic bacterial count of <10 cfu/g as measured by ISO test method no. 4833-1. The substrate body may remain sterile for at least a month, as measured by ISO test method no. 4833-1. The organic fibers may include peat and coir. The rewettable body may retain its hydrophilicity and physical properties while repeatedly fluctuating between a state of low moisture content of as low as 6 wt. % and high moisture content of up to 96 wt. %, based on the weight of the substrate. The substrate body may be generally cylindrical. The substrate body may have a density of about 110-125 kg/m³. The substrate body may have total porosity of about 90-95 vol. %, as measured by Eurofins test method no. FYS1: Cf PTOG nr 31 (1990). The substrate body may have a top surface, a bottom surface, and a body surface extending between and connecting the top surface and the bottom surface, the top surface having a hole having a diameter of about 1-5 mm therein and a depth of about 3-15 mm. The substrate body may be self-supporting. The substrate body may include antioxidants.

In another embodiment, a method for forming a sterile propagation plug is disclosed. The method may include drying a substrate body including a growing medium having bound organic fibers to a moisture content of less than about 20 wt. %, as measured according to ASTM test method no. D2216 and sterilizing the substrate body by exposure to radiation to generate a substantially sterile propagation plug as evidenced by having an aerobic bacterial count of <10 cfu/g, as measured by ISO test method no. 4833-1. The sterilizing may include exposing the substrate body to about 20 to 90 kilograys. The sterilizing may include providing a tray of 100 or more substrate bodies, the substrate body being one of the 100 or more substrate bodies, and exposing the tray of 100 or more substrate bodies to irradiation, the substrate body being disposed in the center of the tray and being confirmed to have a moisture content of below about 20 wt. %, based on the total weight of the substrate body. The tray of 100 or more substrate bodies may include bodies of substantially the same weight. The method may also include air-tight sealing the substrate body after drying and before sterilizing.

In yet another embodiment, a tissue culture tray is disclosed. The tissue culture tray may include a molded container including a plurality of cells, a plurality of sterile, irradiated propagation plugs housed within the plurality of cells, each of the irradiated propagation plugs having a rewettable substrate body including a growing medium having bound organic fibers, the plurality of irradiated propagation plugs having substantially the same dimensions, moisture content of less than about 20 wt. %, based on the total weight of the body, as measured according to ASTM test method no. D2216, and density of about 110-125 kg/m³, according to Eurofins method FYS1: Cf PTOG nr 31 (1990), the plurality of sterile, irradiated propagation plugs being an initial tissue culture medium. The plurality of sterile, irradiated propagation plugs may be hydrophilic. Each one of the plurality of sterile, irradiated propagation plugs may be sterile as evidenced by having an aerobic bacterial count of <10 cfu/g as measured by ISO test method no. 4833-1. Each one of the plurality of sterile, irradiated propagation plugs may retain its hydrophilicity and physical properties while repeatedly fluctuating between a state of low moisture content of as low as 6 wt. % and high moisture content of up to 100 wt. %, based on the weight of the substrate. Each one of the plurality of sterile, irradiated propagation plugs may have substantially the same weight. The tray may also include a flowering plant initiated in each of the plurality of sterile, irradiated propagation plugs, the flowering plant having a system of ground roots.

### BRIEF DESCRIPTION OF THE DRAWINGS

FIG. 1 is a schematic depiction of a prior art tissue culture process;

FIG. 2A is a photograph of a prior art tissue culture medium including agar;

FIG. 2B is a photograph of a plant specimen grown by the prior art tissue culture referenced in FIG. 1;

FIG. 3A is a photograph of non-limiting examples of horticultural plug shapes including the medium disclosed herein;

FIG. 3B is a photograph of a non-limiting example of a tray housing horticultural plugs including the medium disclosed herein;

FIG. 4A is a schematic depiction of a tissue culture process according to one or more embodiments disclosed herein;

FIG. 4B is a comparative schematic of a prior art organogenesis process in 4B1 and a non-limiting example of the herein-disclosed organogenesis process in 4B2;

FIG. 4C is a comparative schematic of a prior art embryogenesis process in 4C1 and a non-limiting example of a herein-disclosed embryogenesis process in 4C2;

FIG. 5 is a photograph of a non-limiting example of a tray with medium disclosed herein;

FIG. 6 is a photograph of a non-limiting example tray disclosed herein;

FIG. 7A is a photograph of a non-limiting example tissue culture medium disclosed herein with a plant supported within the medium, displaying ground or soil roots at the bottom portion of the medium prior to transplanting into a non-sterile medium ex vitro;

FIG. 7B is a photograph of a non-limiting example tissue culture medium disclosed herein configured as plugs supporting plants with soil roots during transplanting stage of the tissue culture process into a non-sterile medium ex vitro;

FIG. 7C is a photograph of the transplanted plants of FIG. 7B; the plants initiated in originally sterile plugs disclosed herein were inserted within a non-sterile medium in a tray;

FIG. 8 is a photograph of a comparative example of anthurium plants grown by tissue culture in agar (on the right) and in a non-limiting example of the herein disclosed sterilized medium (on the left);

FIG. 9 is a water reuptake rate plot for non-limiting examples of the herein disclosed medium of Examples 4A-4C;

FIG. 10 is a water reuptake rate plot for non-limiting examples of the herein disclosed medium of Examples 5A, 5B and comparative examples 5-Ca, 5-Cb;

FIG. 11A is a microscopic photograph of a structure of the comparative example 5-Ca;

FIG. 11B is a microscopic photograph of a structure of the comparative example 5-Cb;

FIG. 11C is a microscopic photograph of a structure of Example 5A;

FIG. 11D is a microscopic photograph of a structure of Example 5B;

FIG. 12A is a photograph of a non-limiting example medium disclosed herein supporting cell culture of somatic embryogenesis;

FIG. 12B is a photograph of the medium of FIG. 12A supporting cell culture of somatic embryogenesis two weeks after initial application; and

FIG. 13 is a water reuptake rate plot for a non-limiting example of the herein disclosed medium of Example 7 and comparative examples 7-Ca and 7-Cb.

### DETAILED DESCRIPTION

Embodiments of the present disclosure are described herein. It is to be understood, however, that the disclosed embodiments are merely examples and other embodiments may take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present embodiments. As those of ordinary skill in the art will understand, various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except in the examples, or where otherwise expressly indicated, all numerical quantities in this description indicating amounts of material or conditions of reaction and/or use are to be understood as modified by the word “about” in describing the broadest scope of the disclosure. Practice within the numerical limits stated is generally preferred. Also, unless expressly stated to the contrary: percent, “parts of,” and ratio values are by weight; the description of a group or class of materials as suitable or preferred for a given purpose in connection with the disclosure implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “substantially,” “generally,” or “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the disclosure can be obtained within a range of +/−5% of the indicated value. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4, . . . , 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. Similarly, whenever listing integers are provided herein, it should also be appreciated that the listing of integers explicitly includes ranges of any two integers within the listing.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

As used herein, the term “and/or” means that either all or only one of the elements of said group may be present. For example, “A and/or B” means “only A, or only B, or both A and B”. In the case of “only A,” the term also covers the possibility that B is absent, i.e. “only A, but not B”.

It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

The term “comprising” is synonymous with “including,” “having,” “containing,” or “characterized by.” These terms are inclusive and open-ended and do not exclude additional, unrecited elements or method steps. The term “including” or “includes” may encompass the phrases “comprise,” “consist of,” or “essentially consist of.”

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed subject matter can include the use of either of the other two terms.

The term “one or more” means “at least one” and the term “at least one” means “one or more.” The terms “one or more” and “at least one” include “plurality” as a subset.

The description of a group or class of materials as suitable for a given purpose in connection with one or more embodiments implies that mixtures of any two or more of the members of the group or class are suitable. Also, the description of a group or class of materials as suitable for a given purpose in connection with one or more embodiments implies that the group or class of materials can “comprise,” “consist of,” and/or “consist essentially of” any member or the entirety of that group or class of materials. First definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

Tissue culture or micropropagation is a growth of tissue or cells in an artificial medium separate from the parent organism. Tissue culture is a biotechnology used to produce clones of a desirable plant. For example, tissue culture has been an important biotechnology implemented to mass produce plants important such as the oil palm, plantain, pine, banana, date, eggplant, jojoba, pineapple, rubber tree, cassava, yam, sweet potato, and tomato. Additionally, tissue culture is used to mass produce new varieties of plants in floriculture and horticulture, especially high value plants such as orchids.

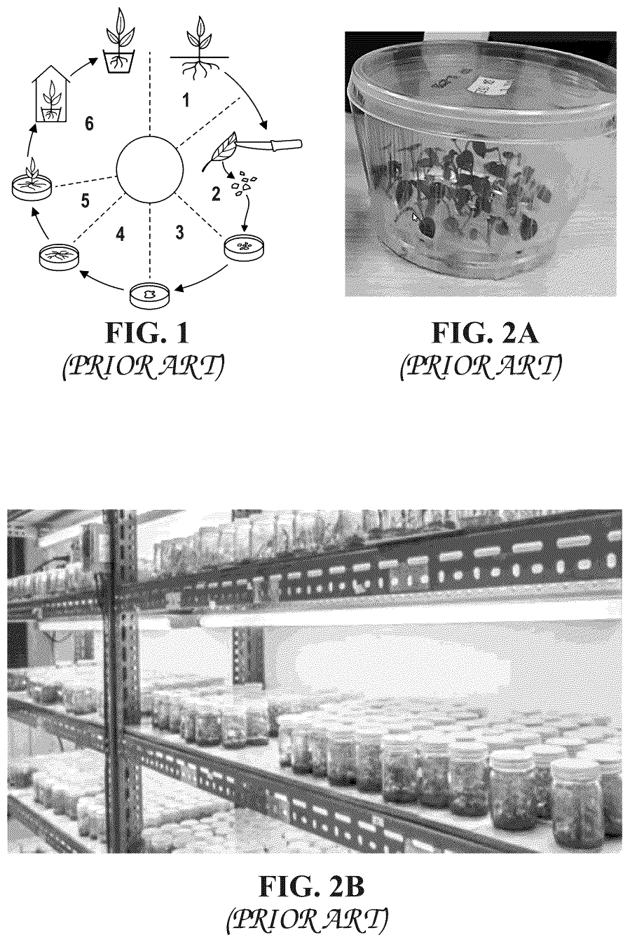

In micropropagation, small pieces of plant tissue are cultured in a nutrient medium under sterile conditions. The following media are typically used for tissue culture: a liquid or gel-like media such as a broth or agar, a seaweed gel. A plant grown by tissue culture transitions through several stages, some of which are in vitro or in a lab environment and some of which are ex vitro or outside of a lab environment. The stages include those captured in the schematic of FIG. 1. As can be seen in FIG. 1, a parent plant is identified at stage 1, and tissue or cells are removed from the parent plant in stage 2 or culture initiation. The removed cells or tissue are placed onto the sterile medium such as agar in a Petri dish in a lab environment. The tissue multiplies, develops, and grows through stages 3 (multiplication) and 4 (rooting) in the Petri dish in the lab environment. At stage 5 or shooting, when the tissue has developed into a young plant having a distinguishable root system and leaves, the plant is transplanted at stage 5. The transplanting presents a number of challenges. Firstly, the plant is taken out of the laboratory environment with relatively ideal conditions such as even temperature and relative humidity, access to nutrients and light, to a real-life environment of a greenhouse. The plant thus experiences a change in temperature, light, and other conditions. Secondly, the plant is removed from the gel-like medium of the Petri dish and placed into a different growing medium such as loose soil or a plug that is not sterile. The removal from the Petri dish may be conducted with tools such as tweezers which may result in damaged or compromised roots and potential damage to the entire young plant. Due to development in agar, the root system tends to be relatively fragile, primarily including water roots which can break easily.

Somatic embryogenesis (SE) is a process by which plants can regenerate bipolar structures from a somatic cell via a tissue culture method. The resulting somatic embryo at the completion of its formation and maturation germinates via elongation of embryo organs (root, hypocotyl, and cotyledon), followed by new shoot growth. This occurs via a step on agar media. Following this step, the plant is moved to soil and acclimatized to the ex vitro environment. Similar to micropropagation benefit of combining stages 4 and 5, combining embryo germination and transfer to a soil media in one step greatly reduces handling and infrastructure, labor costs, and permits greater automation.

Additionally, for both micropropagation and somatic embryogenesis, since the plant was developed in a soft medium such as agar, the root system of such plant can be relatively weak, lacking in fine root hair which are beneficial for survival and thriving in a more robust growing medium the plant is transplanted into. It can take up to a week for the plant to adjust its growth of the root system and start developing root hairs and new lateral roots that are more effective to pull nutrients and water from the new medium. This adjustment slows the overall growth and makes tissue culture more expensive because the plants require a longer time to mature or plants may die, thus taking up valuable space in the labs and greenhouses. FIG. 2A shows a prior art tissue culture growth in agar, resulting in a relatively weak root system.

Furthermore, as the young plant matures into an adult plant, it is typically again translated into a loose soil mix, slab, larger plug, etc. The plant thus faces additional adjustment.

A further disadvantage of the traditional tissue culture lies in the fact that the transplanting can not only be hazardous for the plant, but it is a manual process which is time and man-power consuming. Because the root system of the young plant is very delicate, automation has not been possible. Additionally, plants are grown together in a common Petri dish. Thus, traditional tissue culture requires a lot of careful handling and separation of individual plants, as is illustratively shown in FIG. 2B.

Attempts to utilize non-agar media which could be used from stage 2 to 5/6 without removing the delicate roots from the original growth medium have been made. For example, rubber foam media have been used. But in today's world, bio-degradable and bio-derived materials are sought, and in some cases already required by various regulations. Hence, the rubber foam media may not be acceptable. Yet, providing a bio-based or organic material-based medium which could serve as a medium both in vitro and ex vitro comes with a plethora of problems which have not yet been solved.

It has been discovered that a wet plug cannot be adequately sterilized effectively or cost effectively. Additionally, traditional plugs, such as those based on peat, become hydrophobic below about 20 wt. % moisture content. A hydrophobic plug is unsuitable for plant growth. Drying below 20 wt. % may eliminate surface tension such that moisture does not adequately enter the plug and the plug remains relatively dry, essentially not allowing penetration of liquids within the plug, thus not adequately supporting transport of water and nutrients to a plant located within the plug. Additionally, some of the traditional sterilization processes may damage or alter the structure of the plug.

Thus, there is a need to develop an organic material-based medium for both in vitro and ex vitro environments (and transition from in vitro to ex vitro environments) for micropropagation or tissue culture of various plants.

In one or more embodiments, a micropropagation medium is disclosed. The medium may be used for tissue culture, organogenesis, somatic embryo development, or other growth of plants requiring initiation in a laboratory, sterile environment.

The medium may be suitable for both in vitro or lab environment and ex vitro or outside of lab environment such as a greenhouse or outdoors conditions. The medium may be configured as a vehicle for in vitro to ex vitro plant growth, specifically micropropagation. The medium may be suitable for growth of horticultural and floricultural plants including, but not limited to, ornamental plants, fruit-bearing species, trees, flowering plants, berry plants, medicinal plants, etc. Non-limiting examples of the suitable plants may include banana, potato, sugarcane, apple, pineapple, strawberry, gerbera, anthurium, lilium, orchid, bamboo, date palm, teak, pomegranate, venus flytrap, rubber tree, cassava, yam, or the like. The medium may be suitable for growth of new varieties.

The medium may be structured as a plug, growing medium plug, horticultural plug, or a starter plug. A plug is a compact mass of a growing medium, typically used for seed germination and rooting cuttings. The plug may be self-supporting. The term “self-supporting” refers to the plug's ability to exist as a cohesive body manipulable as an intact entity.

The plug may have any shape, size, or configuration. A non-limiting example shape may include a full or partial cylinder, cube, prism, cone, hemisphere, bullet, sphere. The plug may be a reverse pyramid, cone, or the like. The plug may have a body having a uniform or non-uniform shape. The plug may have one or more sides. The plug may have a top surface, a bottom surface, and a body surface extending between and connecting the top surface and the bottom surface.

The body surface may include one or more indentations, protrusions, ridges, lines, dents, the like, or a combination thereof. The body surface may include one or more tapers. For example, in a non-limiting example, the body surface may include one or more walls tapered towards the bottom.

The top and bottom surfaces may have the same or different size, shape, configuration, or a combination thereof. The top surface may include a low angle cavity or depression or hole having a diameter of about 1-5 mm therein and a depth of about 3-15 mm. The diameter may be about, at least about, or at most about 1, 1.5, 2, 2.5, 3, 3.5, 4, 4.5, or 5 mm. Other dimensions are contemplated. The depth may be about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mm. Other dimensions are contemplated. Alternatively, the top may include a thumb print or a shallow dent instead of a hole. The top may also be free of any hole, dent, protrusion, cavity, or depression.

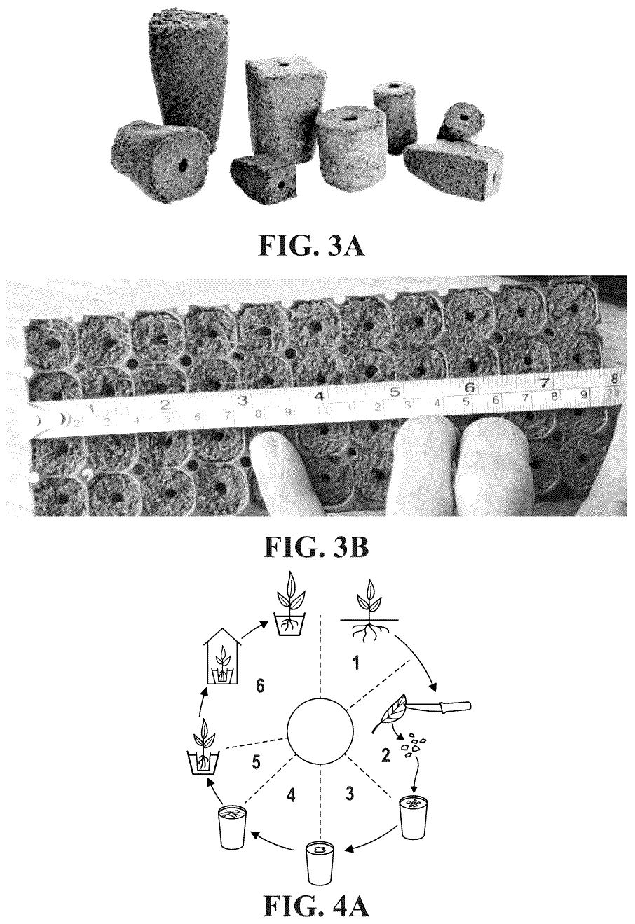

Non-limiting examples of the plugs are shown in FIGS. 3A and 3B. FIG. 3A shows various plug shapes and sizes. FIG. 3B shows a number of non-limiting example plugs provided in cells of a non-limiting example tray. The plugs may have a dibble, indentation, recess in the top side of the plug, as is shown in FIGS. 3A and 3B. Alternatively, the plugs may be free of a dibble, indentation, or recess.

The medium is rewettable. The medium may be rewettable at least one time. The medium may be rewettable repeatedly. The term “rewettable” refers to the ability of the medium to be dried to a certain moisture content and then moistened again to a degree which allows a liquid to be absorbed into the plug. The rewettability is repeated such that the medium's moisture content may fluctuate without the plug losing the ability to wick or absorb water. The medium may thus retain its hydrophilicity and/or physical properties while repeatedly fluctuating between a state of low moisture content of as low as about 6 wt. % and high moisture content of up to about 80, 90, or 96 wt. %, based on the weight of the substrate, for example during use in tissue culture. The high moisture content may be up to about 80, 81, 82, 83, 84, 85, 86, 87, 88, 89, 90, 91, 92, 93, 94, 95, or 96 wt. %, based on the weight of the substrate at field capacity which relates to stable weight after media is allowed to drain after irrigation.

The medium may have an initial moisture content above about 15-20 wt. %, be dried to decrease moisture to a secondary moisture content of below about 15-20 wt. %, and then may be rewettable to increase moisture to a tertiary moisture content of above about 20 wt. % during micropropagation use. In other words, the secondary moisture content is less than the initial and tertiary moisture contents, which may also differ from each other. The medium may have a moisture content of about or at most about 6-20, 10-18, or 12-15 wt. % before use in a laboratory environment (initial moisture content, secondary moisture content, moisture content before irradiation, during irradiation, after irradiation, in storage, or a combination thereof). The plug may have a moisture content of about, at least about, or at most about 6, 7, 8, 9, 10, 11, 12, 13, 14, 15, 16, 17, 18, or 19 wt. % during irradiation, before propagation use, or both. The moisture content during irradiation may be about 6-19, 8-16, or 9-12 wt. %. The moisture content of the medium should be such that the medium may be rendered sterile by the process described herein and also rewettable for propagation use. Moisture content within this disclosure may be assessed using ASTM method D2216 or alternatively using an automatic program utilizing a moisture analyzer such as Mettler Toledo HE73. The automated program uses a halogen bulb in a confined analysis chamber to heat a sample to 160° C. under constant mass analysis. The sample is heated until the mean weight loss is less than 1 mg per 30 seconds, which typically takes between 5 and 15 minutes, depending on initial moisture content. After this, moisture content is determined by the following formula:

$$Moisture Content = \frac{initial weight - dry weight}{initial weight} \cdot 100 \% .$$

The medium is hydrophilic. The medium may be hydrophilic despite fluctuations of its moisture content. The medium may retain is hydrophilicity through its lifetime. The medium may be hydrophilic, but surfactant free. While one or more surfactants may be used to render the medium hydrophilic, the medium may be hydrophilic without inclusion of a surfactant. Thus, the medium disclosed herein may be hydrophilic, but not including a surfactant. In one or more embodiments, the medium may include a surfactant in an amount not negatively affecting plants.

The medium is sterile. The medium may thus be free of microorganisms such as bacteria, viruses, fungi, yeast, the like, or a combination thereof. The bacteria may be Gram-negative, Gram-positive, aerobic, anaerobic, or a combination thereof. The medium may be sterile according to ISO 16649-2 (E. coli), ISO 6888-1 (Staphylococcus aureus), ISO 4833-1 (aerobic bacterial count), EMGS (fungi/yeast), the like, or a combination thereof. The medium may retain sterility for a predetermined amount of time or until use. The medium may be sterile for several hours, days, weeks, months, or years. The sterility may be complete sterility. The sterility may be high such as all pathogenic organisms are removed. The degree of sterility may be such that spores and DNA of any microorganisms are eliminated, the medium contains no dormant spores, microbe DNA, or a combination thereof.

To render the medium sterile, the medium may be irradiated. The radiation may be an ionizing radiation. The radiation may be gamma radiation. Ionizing radiation is a form of energy that acts by removing electrons from atoms and molecules of materials that include air, water, and living tissues. Due to high penetration of the ionizing radiation, microorganisms, and their spores and DNA, can be eliminated. At the same time, such ionizing radiation is used that does not significantly alter physical structure, chemical properties, or both of the medium or may leave the physical structure and chemical properties of the medium intact. Using an increased dose of the irradiation may be non-economical and/or result in the plug being attacked by radicals generated during the irradiation process to such a degree that the structural stability and other properties of the plug may be jeopardized.

While other types of sterilization processes are known, such as autoclaving, microwaving, chemical treatment, or the like, such sterilization is typically not sufficient for micropropagation purposes because spores may survive high heat and various sterilization chemistry. The traditional processes are also typically costly and labor intensive. Autoclaving can also result in non-sterile media if temperature and pressures are not correctly assessed in the autoclaving chamber. Likewise, sanitation or pasteurization are generally not suitable as they do not eliminate the presence of fungal and bacterial spores. The surviving spores may then activate in the in vitro environment and thrive in the medium instead of the desirable cell culture. Additionally, some of the processes, including autoclaving, may not be suitable for tissue culture plugs as the processes may physically damage the plugs.

The medium material may be bio-based, predominantly or completely organic material-based, bio-degradable, home compostable, industry compostable, or a combination thereof. The medium may be fibrous. The medium may be hydroponic. The medium may be soil-less or soil free. The medium may be solid, non-gel like, not semi-solid.

The medium material may include one or more types of fiber. The fiber may include natural fiber such as peat, coco coir, wood fiber, bark fiber, cotton, hemp, flax, sisal, jute, kenaf, bamboo, wheat, the like, or a combination thereof. The fiber may be lignin-derived, lignocellulosic fiber. The fiber/medium may include natural or added antioxidants, radical inhibitors, radical quenchers, the like, or a combination thereof. A non-limiting example medium may include a combination of peat fiber and coco coir fiber, in a ratio of 1:9-9:1. Other non-limiting example media may include a peat fiber or coco coir fiber, or combinations of peat fiber and coco coir fiber in a ratio of 1:8-8:1, 1:7-7:1, 1:6-6:1, 1:5-5:1, 1:4-4:1, 1:3-3:1, 1:2-2:1, or 1:1. The coco coir fiber may be buffered, or otherwise adjusted, to minimize salt content.

The medium may include a binder. The binder may be based on natural or synthetic compositions. The binder may be poly-urethane based. The binder may be toluene diisocyanate-based (TDI) or non-TDI-based. Other suitable polymeric and non-polymeric binders may also be used. The binder may include radical inhibitors, radical quenchers, the like, or a combination thereof.

The medium may include additional materials such as surfactant(s), biodegradable synthetic fibers, plant hormone(s), fertilizer(s), macronutrient(s), micronutrient(s), mineral(s), mineral particle(s), heat-treated mineral particle(s), the like, and/or combinations thereof. The material may be free of one or more of surfactant(s), biodegradable synthetic fiber(s), hormone(s), fertilizer(s), macronutrient(s), micronutrient(s), mineral(s), natural gum(s), heat-treated mineral particle(s), the like, and/or combinations thereof.

Surfactants may include one or more surfactants in a mixture or blend. Surfactants, or surface-active agents, are compounds that lower the surface tension between two liquids or between a liquid and a solid. Surfactant(s) may be added to increase foaming, hydrophilicity, void formation, bubble formation and stability, structural stability of the final product, the like, or a combination thereof. A non-limiting example surfactants may include anionic surfactants, cationic surfactants, zwitterionic surfactants, non-ionic surfactants, amphoteric surfactants, or their combination. Surfactant(s) may be included in an amount of about 0.01 to 5 vol. or wt. %, based on the total volume or weight of the medium or the slurry. The surfactant amount may be about, at least about, or at most about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9. 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5 vol. or wt. %, based on the total volume or weight of the medium or the slurry.

Biodegradable synthetic fibers may include fibers of cellulose or other materials.

Plant hormones are signal molecules that control various aspects of plant growth and development. Non-limiting example plant hormones include auxins, cytokinins, gibberellins, ethylene, abscisic acid, the like, and/or combination thereof.

Fertilizers such as nitrogen fertilizers, phosphate fertilizers, potassium fertilizers, compound fertilizers, and the like may be used. For example, melamine/formaldehyde, urea/formaldehyde, urea/melamine/formaldehyde and like components may serve as a slow-release or control-release fertilizer. Fertilizers having lesser nutritional value, but providing other advantages such as improving aeration, water absorption, or being environmental-friendly may be also used. The source of such fertilizers may be, for example, animal waste, compost, and/or plant waste. Control-release fertilizers with polyolefin, polyurethane, polymeric, bio-based, and/or biodegradable coating may be incorporated into the medium.

Nutrients may include macronutrient, micronutrients, and minerals. Examples of macronutrients include calcium, chloride, magnesium, phosphorus, potassium, and sodium. Examples of micronutrients are also well-known and include, for example, boron, cobalt, chromium, copper, fluoride, iodine, iron, magnesium, manganese, molybdenum, selenium, zinc, vitamins, organic acids, and phytochemicals.

The mineral particle(s) may include perlite, vermiculite, sand particles, zeolite, hydrated aluminosilicate minerals that contain alkali and alkaline-earth metals, or a combination thereof. The mineral particle(s) may be treated or untreated.

The heat-treated mineral particles may include calcined particles. The calcined particles may be based on clay. The calcined clay particles may include one or more types of clay. The clay may include, for example, smectite clay(s) including the following minerals: montmorrilonite, beidellite, nantronite, saponice, hectorite. The clay may be gray, red, or both. The clay particles may be processed in the following manner for the purposes of the disclosed application. The clay may be calcined at a temperature of about 1000 to 1400, 1100 to 1350, or 1200 to 1300° F. or 537 to 760, 593 to 732, or 648 to 704° C. The clay may be subsequently sized or micronized, for example, by grinding. The clay may be provided in various sizes.

The medium disclosed herein may have density of about 100-135, 110-125, 112-120, or 115-119 kg/m³, according to FYS1: Cf PTOG nr 31 (1990) method by Eurofins. The density may be about, at least about, or at most about 100, 101, 102, 103, 104, 105, 106, 107, 108 109, 110, 111, 112, 113, 114, 115, 116, 117, 118, 119, 120, 121, 122, 123, 124, 125, 126, 127, 128, 129, 130, 131, 132, 133, 134, or 135 kg/m³, according to FYS1: Cf PTOG nr 31 (1990) method by Eurofins. Bulk density is the weight of 1 m³ dry product. In the method, each sample is wetted to different pressure heights, the dry weight is expressed in kg/m.

The medium is porous. The porosity may be relatively or substantially uniform throughout the mass of the medium body. The medium disclosed herein may have total porosity or volume of fraction of pores of about 90-95, 90.5-94.5, or 91-94 vol. %, according to FYS1: Cf PTOG nr 31 (1990) method by Eurofins. The pores are the column in the substrate that contains air. Bulk density and organic matter indicate the solid fraction. The total volume minus the solid fraction are the pores, as expressed in vol. %, based on the total volume of the medium. The total porosity may be about or at least about 90, 90.5, 91, 91.5, 92, 92.5, 93, 93.5, or 94 vol. %, according to FYS1: Cf PTOG nr 31 (1990) method by Eurofins.

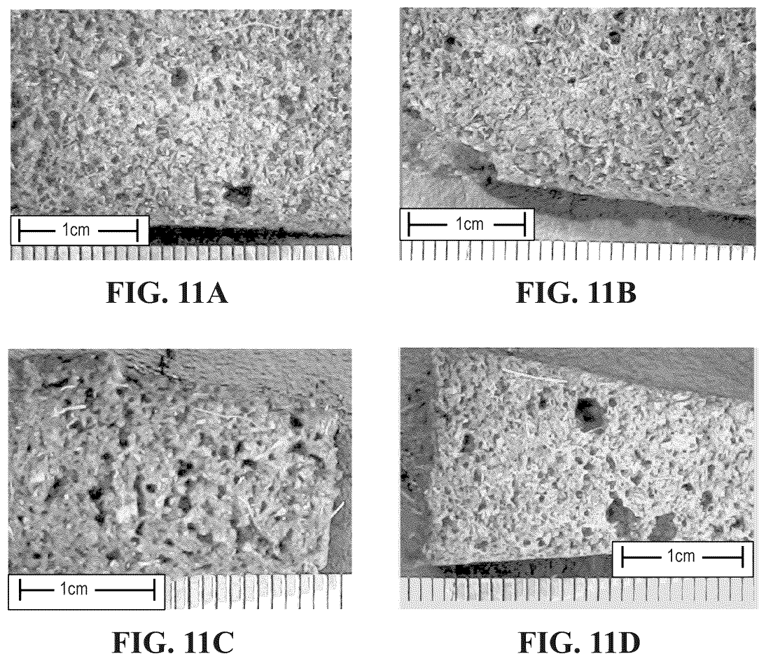

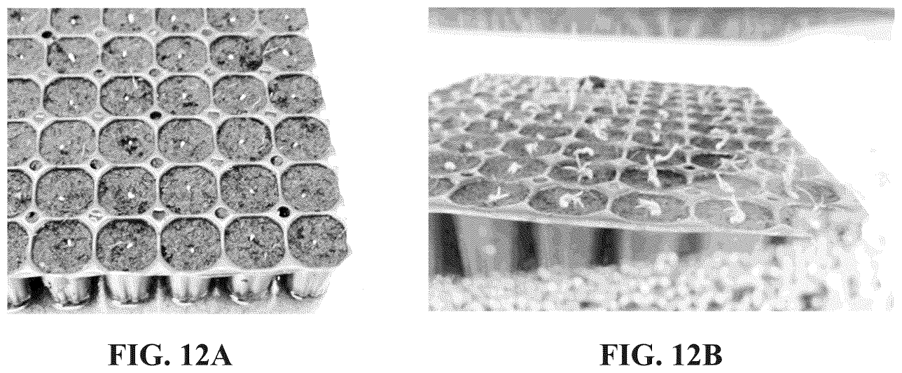

The disclosed porosity enables relatively balanced distribution of air and water throughout the medium. The porosity influences the medium's ability to rewet, absorb water repeatedly, and repeatedly dry, and thus support absorption of nutrients for the culture being propagated within the medium. Non-limiting examples of the porous nature of the herein-disclosed medium is shown in FIGS. 11C and 11D.

The medium in a loose form or as self-supporting plugs may be inserted into cells of a tray. The tray may be a container with a plurality or number of cells. The cells may be molded or otherwise formed in the surface of the container. The cells may be spaced apart in a regular or irregular pattern. Typically, the cells are organized in rows, have the same dimensions, shape, and configuration. Each cell is structured to impart a desirable shape to the plug to be formed within the cell. Each cell has one or more walls terminating in a bottom, flat side, forming a distal end with respect to the container surface. The one or more walls also include a rim from which the one or more walls protrude away from the surface. A non-limiting example profile of the cell may include a cuboid, cylinder, upside down flat top pyramid, multi-angle prism such as a pentagonal or hexagonal prism, cone, upside down flat top cone, or the like. A cell may have a changing cross-section throughout its length. In a non-limiting example, the cross-section at the rim may be a square and the cross-section of the bottom may be a circle for the same cell. A cross-section may be a circle, square, rectangle, pentagon, hexagon, oval, or the like. The walls and the rim may have rounded edges. The walls may be smooth. In some embodiments, at least one of the walls may include indentations, ridges, lines, or other protrusions or dents.

The cells may be indentations in a main body of the tray. The cells may be shaped as a well, deep recess, open-top container, into which a medium may be inserted. The cells can include a bottom, sides, and an open top. The cells may be uniform or non-uniform, having the same or different dimensions such as height, width, or depth. The cells may form a housing space for the plugs or loose medium which may form a self-supporting structure once the root system develops within the medium of a cell.

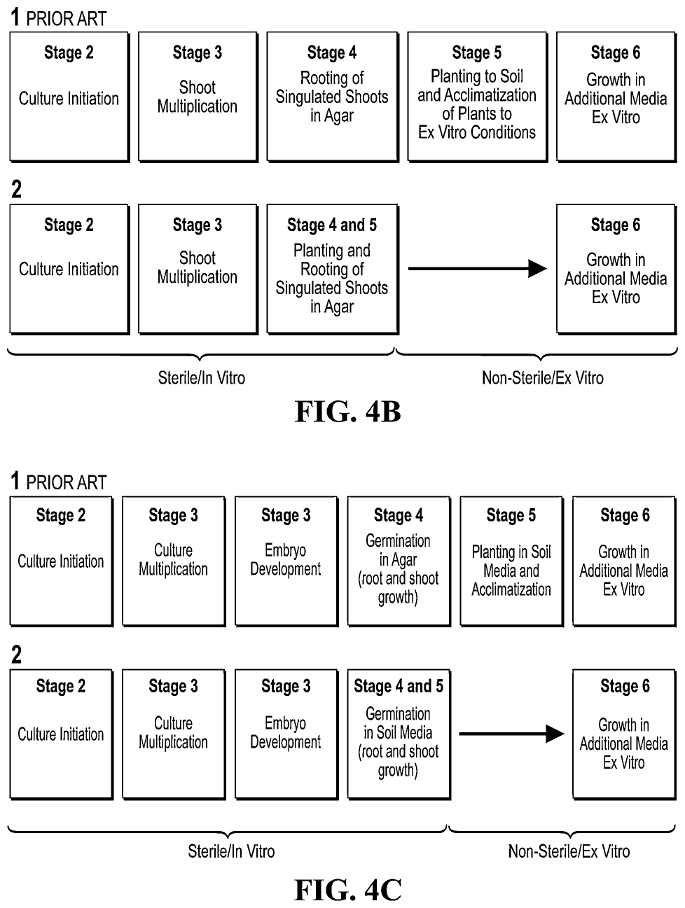

The tray may have various dimensions. The tray may be dimensioned to fit the needs of a radiation equipment, laboratory spatial requirements, or both. A non-limiting example of a suitable tray is shown in FIGS. 3B, 5, 6, 12A, and 12B. The tray's dimensions and number of cells may be varied as desired. The material of the tray should be suitable for the drying step, radiation exposure, and propagation through stages 1-4 of the cycle shown in FIG. 4A.

The tray and the medium inserted within the tray cells may be part of a kit. The kit may include the medium as loose material inserted in the cells or plugs inserted in the cells, the medium being dried and irradiated. The tray with the irradiated medium may be placed into a sterile packaging for transport. The kit may be suitable for micropropagation, somatic embryos propagation, or other types of propagation which require initiation in a lab environment.

The medium disclosed herein may be the first or initial medium of the plant growing process, tissue culture, or somatic embryogenesis starting with the planting/rooting stage. As the plant matures, the plant rooted in the initial sterile medium may be transplanted into a non-sterile secondary, tertiary medium, or additional medium while continuing to be rooted in the initial medium. The plant thus retains direct contact with the initial medium throughout its life. Throughout the plant growth and life, the plant may remain rooted in the initial medium, its roots expanding through the initial medium into the additional media.

A process for making the in vitro to ex vitro medium is disclosed herein. The one or more type of fiber and one or more solid additional materials named herein may be mixed together to form a mixture. Subsequently, the binder and optionally any surfactants may be added to the mixture to form a slurry. The slurry may be provided into the trays or molds to form the medium. The medium is subsequently set or cured. An optional dibble may be provided in an automated manner. Trays with the medium may be then stacked, wrapped, stored, or a combination thereof.

Before irradiation, in at least one embodiment, the medium is dried to the moisture content disclosed herein, i.e., below or to 20, below 18, or below 15, but above 5.9 or 6 wt. %, based on the total weight of the medium/plug. The moisture content achieved in the drying process may be about 6-19, 8-16, or 9-12 wt. %. The moisture content achieved in the drying process may be about, at most about, or at least about 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, or 19 wt. %. A range utilizing any two numerals disclosed herein is contemplated.

The drying may be done in ambient air or in an oven, or in other ways. For the drying step, the medium may be inserted into a tray and dried together. Alternatively, individual plugs or containers with the medium may be dried individually. The drying may be provided by a slow-drying process. The drying may last for several hours to several days such as about 2-20 days, 4-15 days, or 6-14 days. In a non-limiting example, the medium may be shaped into plugs, inserted into a tray as plugs, or inserted into a container such as a tray in a loose form before or after for the drying step. The drying may be done relatively uniformly such that a plurality of plugs have substantially the same moisture content of below 20 wt. % after the drying step and before irradiation. The relatively uniform drying may be achieved, for example, by relatively uniform air circulation in a drying chamber.

The dried plugs may be wrapped in a packaging following the drying process. The packaging helps the medium to retain or maintain the desirable moisture content and prevents fluctuation of the moisture content before irradiation. In at least one embodiment, the process thus includes achieving a desirable moisture content disclosed herein and maintaining the moisture content up until and throughout irradiation. The process results in having a plurality of plugs, trays, or both with uniform moisture content within the ranges disclosed herein. The packaging may be made from a polymeric material. The packaging may be air-tight. The dried medium may be sealed within the packaging.

Irradiating plugs having a higher moisture content than 20 wt. % may result in the plugs not being sterile or losing sterility within an undesirably short period of time (days or weeks). Additionally, a higher moisture content than 20 wt. % contributes to and increases the mass of the plugs, which in turn requires a higher dose of irradiation. Using higher irradiation doses results in higher power consumption required for irradiation and increased radical formation during irradiation, which may compromise structural integrity of the plugs and leads to an accelerated destruction of the plugs.

Additionally still, a moisture content which is too low (below 6 wt. %) may result in a plug which is non-rewettable or requires a long time to rewet. A non-rewettable medium is generally not suitable for propagation. An over-dried plug is typically soaked in a liquid to reabsorb moisture. This results in a loss of uniformity between the plugs within a tray or trays which is problematic. Nutrition in the tissue culture is provided strictly via an added nutrient liquid by the lab technician. The amount of the nutrient liquid added to each plug should be precise and is typically applied as the same amount of liquid for each plug in a tray. A uniform substrate is thus required to ensure that the applied nutrient liquid amount is suitable. If the substrate is non-uniform with respect to moisture content and liquid absorption, each plug will absorb the added nutrients differently, and the lab cannot efficiently utilize the plugs.

The herein-disclosed substrate thus features a plurality of sterile plugs having uniform moisture content throughout the entire or substantially entire plurality. This in turn enables precise administration of nutrients to each plug, predictable uptake of the nutrients within the plugs, and thus predictable growing results among the propagated plants.

After the medium is dried, the medium is exposed to radiation. The amount of radiation may be about 20-90, 30-70, or 40-50 kilo gray. The amount of radiation may be about, at least about, or up to about 20, 25, 30, 35, 40, 45, 50, 55, 60, 65, 70, 75, 80, 85, or 90 kilo gray or a range including any of the disclosed numbers. 1 gray of GY is 1 Joule/kilogram, which equals to 100 rad. The amount of irradiation is determined based on weight of the medium including any trays, pellets, boxes, packaging, etc. which are provided to the irradiation facility.

The ratio of irradiation dose to the medium moisture content may be about 20-90 kGy to 6-20 wt. %. The ratio may be a range from 1 to 15, 3.3 to 10, or 4.5 to 8. The ratio may include values of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 kGy:wt. %.

Subsequently, the process may include placing the irradiated medium in storage and/or use for tissue culture, somatic embryos propagation, the like, or a combination thereof. The process may include packaging the irradiated medium, for example in a sterile packaging material. The packaging material may be vacuum sealed, be airtight, or both. The method may include retaining the sterile medium in a sterile container such as a tray for a number of hours, days, months, or years before use. The medium may retain its sterility for at least a few or several hours, days, months, or years before use.

A process of using the medium is disclosed herein. The process may include applying a liquid such as an aqueous nutrient liquid onto the medium to render the medium moist, placing a plant cell material onto the moistened medium, distributing the liquid and/or cells evenly, propagating the plant on/in the medium in vitro, ex vitro, or both, once the plant has roots and leaves, taking the plant rooted in the medium and placing the medium into a secondary medium, placing the secondary medium with the plant rooted into the first medium into a tertiary medium, or a combination thereof. The process may be free of removing the initial medium from the roots, exposing roots to air, transplanting roots without the initial medium, or a combination thereof.

The process may include robotized or automated use of the medium. For example, the process may include robotized tissue culture or micropropagation. The process may include one or more automated steps of the tissue culture cycle such as cutting plant material, placing the cut plant material onto the irradiated material/plug, repeating the cutting and placing for each cell of a tray, or a combination thereof. Furthermore, the process may include one or more automated steps such as transplanting the plant at stage 5 into a secondary medium, sorting the transplants, or both.

The advantages of the herein-disclosed medium may include an altered, simplified, and accelerated micropropagation cycle. The improved process is shown in FIG. 4A. FIG. 4B provides a comparison of the prior art organogenesis process (FIG. 4B1) with the herein-disclosed organogenesis process (FIG. 4B2). FIG. 4C provides a comparison of the prior art embryogenesis process (FIG. 4C1) with the herein-disclosed embryogenesis process (FIG. 4C2).

While the initiation, multiplication, and rooting formation steps of the traditional tissue culture remain, the transplanting step, which exposes roots and removes the initial medium from the plant, is eliminated. Instead, the medium disclosed herein serves as the initial, on-going, and lasting medium throughout all micropropagation stages. All steps of the micropropagation may thus be conducted in the sterile medium disclosed herein.

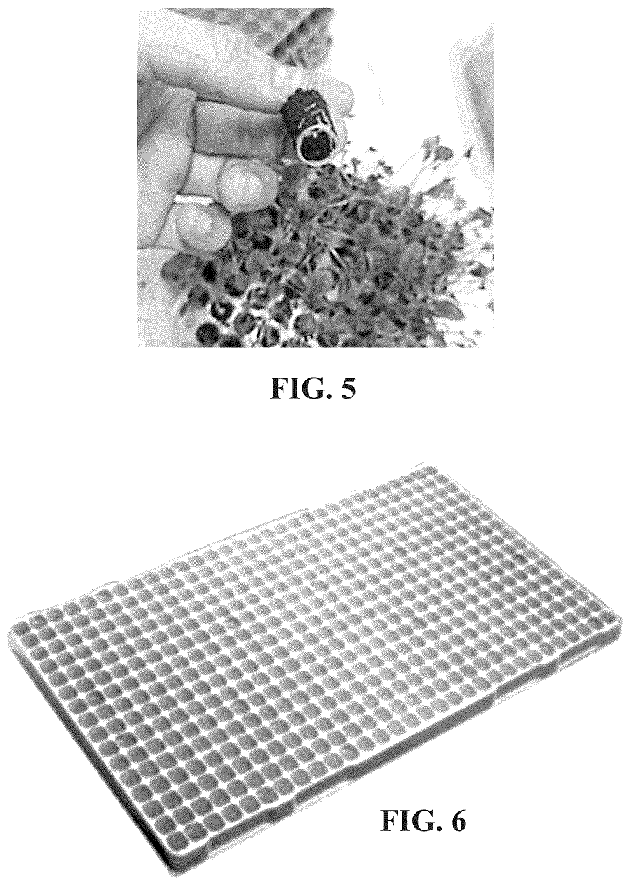

The inserted medium may be loose or shaped as plugs, inserted in individual cells of a tray. A non-limiting example of such medium is shown in FIG. 5. FIG. 5 shows the medium shaped as a non-limiting example micropropagation plug used for organogenesis of bellwort. At the stage 5, when the plant has roots and leaves, the medium is not removed from the plant roots. Instead, the plant with roots intact remains in the medium. The medium including the plant rooted within the medium may be removed from the tray and placed into a secondary growing medium such as a potting soil in a container, slab, or the like. The roots may continue growth through and from the plug/medium into the secondary medium without being cut, damaged, or exposed to loss of medium. The structure of the roots and plant may thus remain intact, non-desirable impact may be thus avoided.

An additional advantage is that the plug is not a soft, gel-like medium such as agar, as shown in FIG. 2A. Hence, all or a majority of the roots of a plant propagated in the herein-disclosed medium, are ground or soil roots. The ground or soil roots differ from the above-mentioned water roots, which typically develop in soft media such as agar. Water roots are typically very thin with a lot of offshoots, providing an almost hairy appearance. While the water roots grow faster and require less space than the ground roots, they are fragile and susceptible to breakage. Since they develop in the soft agar, their physical nature and appearance stems from lack of need to search for nutrients as nutrients are readily provided and easily reachable.

In contrast, the ground or soil roots are typically less thin, sturdier, with many root hair endings and lateral roots. Their sturdiness protects them from breakage. The ground or soil roots may include fine fuzzy endings or root hairs which are capable of searching for and taking in nutrients, air, and water. The soil roots are better adjusted for survival outside of a lab environment and faster growth than the water roots.

As was mentioned above, in a traditional tissue culture, the ground roots do not start developing until stage 5 when the plant is transplanted out of agar, and the plant is forced to develop ways of reaching nutrition. In the herein-disclosed medium, such adjustment is avoided, as the plant starts developing the soil roots from the time the first roots emerge. The overall plant growth is accelerated, up to about 50-60% based on an average in comparison with the traditional tissue culture plant growth.

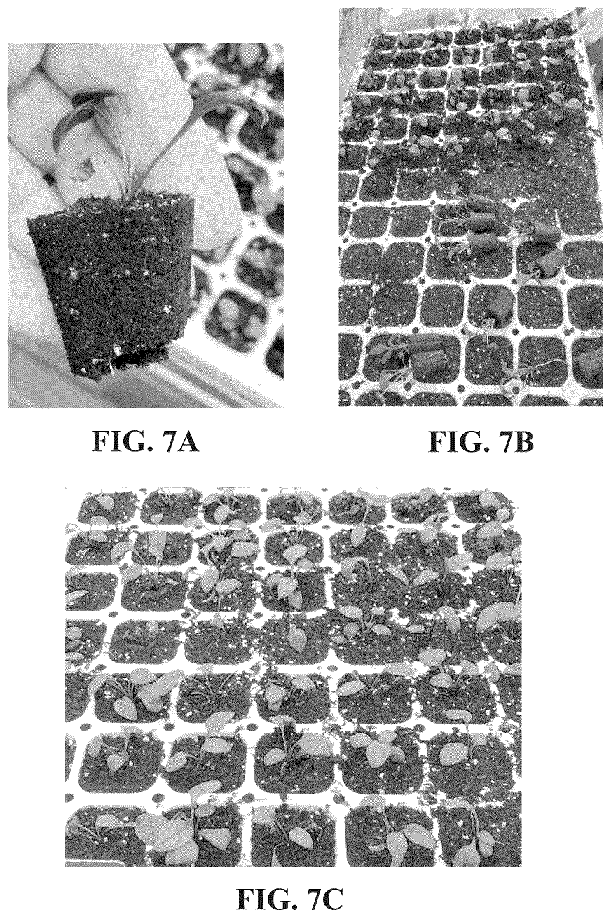

A non-limiting example plants developed by tissue culture as disclosed herein are shown in FIGS. 7A-7C and described below as Example 2. As can be observed from FIG. 7A, the plant has developed thin diameter ground root system, reaching to the bottom of the plug, before being transplanted into trays with loose, non-sterile medium shown in FIGS. 7B and 7C in a greenhouse setting.

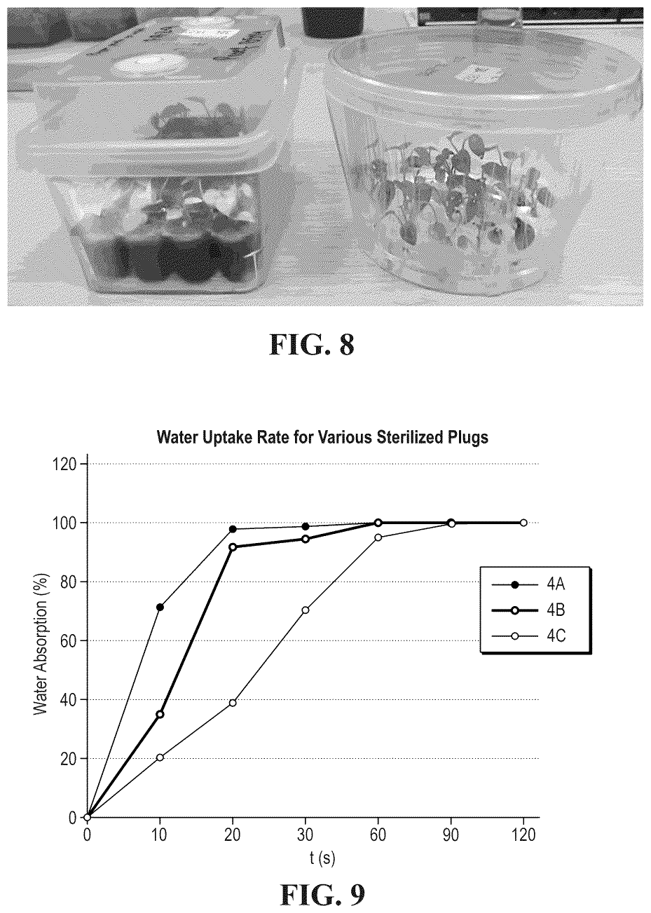

An additional non-limiting example is a comparison growth of anthurium initiated in agar and in the herein-disclosed medium, respectively, shown in FIG. 8 and described as Example 3. Anthurium grown in agar is depicted on the right; the plants cultured in agar having a weaker stem and weak root system in comparison to the anthurium grown in the sterilized medium disclosed herein shown on the left. The anthurium propagated in the herein-disclosed medium showed accelerated growth, stronger plant structure, and sturdier root system.

Further still, because the sterilized medium disclosed herein may be inserted into trays, the micropropagation process may be automated or robotized, the amount of handling may be minimized, which may result in a more economical and efficient micropropagation.

### EXAMPLES

### Example 1

140 trays with samples of the medium disclosed herein in the tray cells were prepared by a method described above. The trays included peat-based samples including mixtures of peat and coir. The samples were dried for 3 or 6 days, respectively. The samples were exposed to ionizing radiation in the trays in March 2023 and then again in March 2024. The test measured presence of various microorganisms listed in the table below. All the samples in the 140 trays were found to be sterile. The sterility was verified a year after initial irradiation and storage.

TABLE 1
Samples of Example 1 sterility test results

| | No. of | | | Result in | Results in |
|---|---|---|---|---|---|
| | drying | | | March 2023 | March 2024 |
| Samples | days | Test method | Microorganism | [cfu/g] | [cfu/g] |
| A | 3 | Lab method | Aerobic bacterial counts | <100 | <100 |
| | | | 30° C. | | |
| | | Lab method | Plate count fungi/yeast | <10 | <10 |
| | | ISO 16649-2 | Escherichia coli | <10 | <10 |
| | | ISO 688-1 | Staphylococus aureus | <10 | <10 |
| B | 6 | Lab method | Aerobic bacterial counts | <100 | <100 |
| | | | 30° C. | | |
| | | Lab method | Plate count fungi/yeast | <10 | <10 |
| | | ISO 16649-2 | Escherichia coli | <10 | <10 |
| | | ISO 688-1 | Staphylococus aureus | <10 | <10 |

### Example 2

Several trays of coir, peat mix plugs were prepared, dried, and irradiated according to the description provided herein. The drying included bringing the moisture content of the plugs to the level of about 9-12 wt. % and packaging the trays after drying and before irradiation. The irradiation was conducted at about 35 to 50 kGy. The trays were tested in tissue culture settings. The plugs showed retained sterility and the plants showed good growth. The growth was substantially uniform throughout the trays. FIG. 7A shows an example plant with developed root structure in a plug, with soil roots protruding from the bottom of the plug, indicating a healthy root development of the plant initiated within the plug. FIG. 7B shows a number of plugs with a strong root system developed in the sterilized plugs disclosed herein before the plugs were replanted into a non-sterile loose medium in a tray. FIG. 7C shows the plants replanted at stage 5 of the cycle described herein after cultivation in the sterilized plugs. The plants showed uniform growth and soil root development throughout.

### Example 3

Example 3 was set up to compare viability of the herein-disclosed medium for tissue culture compared to traditional agar use. A tray of 12 plugs of the herein-disclosed medium containing both coir and peat fiber was prepared by a process described herein. The plugs were subsequently dried to the moisture content between about 9 and 15 wt. % and sterilized by gamma radiation set at about 35-50 kGy. The plugs of the tray were subsequently provided with tissue culture of anthurium, which was propagated in typical tissue culture conditions.

A comparative set of tissue culture anthurium samples was initiated in agar and propagated under the same conditions as the cuttings in the tray.

At the time the photographic image of FIG. 8 was taken, the plants were assessed. The plugs remained sterile, thus carrying no living organism expect for the plants. The plants in the agar showed weaker root system, stem, and leaf development compared to the plants grown in the tray.

### Example 4

Example 4 was set up as a trial to test rewettability of herein-disclosed plugs. Six trays with 12 plugs each were prepared. The plugs were prepared according to the method described above, dried before irradiation, and irradiated at about 35-50 kGy. The composition of the plugs is shown below in Table 2.

TABLE 2
Composition and properties of media of Example 4

| Example | | Binder [wt. % | | Surfactant | Sterilized by |
|---|---|---|---|---|---|
| No. | Binder | per slurry] | Composition | amount [wt. %] | irradiation |
| 4A | TDI | 0.84 | 70:30 | 0.2 | Yes |
| | | | (coir:peat) | | |
| 4B | TDI | 1.2 | 70:30 | 0.2 | Yes |
| | | | (coir:peat) | | |
| 4C | TDI | 2.4 | 70:30 | 0.2 | Yes |
| | | | (coir:peat) | | |

Two trays of each example 4A, 4B, and 4C were set in a flat-bottomed, steel basin filled with 4 mm of water. At set intervals (10s, 20s, 30s, 60s, 90s, and 120s), a tray was removed from the basin and weighed. Afterwards, the trays were all soaked in water until fully saturated (12 min). The relative rate of rewetting is shown in FIG. 9. FIG. 9 shows a relative water uptake rate of the Examples 4A-4C. The x axis is non-linear.

### Example 5

Water reuptake rates were assessed for the medium disclosed herein in comparison to competitive media. All the tested plugs had substantially the same shape and size. Example 5A and 5B are plugs of the medium disclosed herein, prepared according to the process described above. The plugs of 5A and 5B included coir, peat, binder, and surfactant as described above. Both plugs 5A, 5B had moisture content of 15.2 and 14.9 wt. %, respectively, and were subsequently irradiated as described above.

Comparative examples 5-Ca and 5-Cb had similar composition as 5A and 5B regarding fiber content. The moisture content of 5-Ca was 16.3 wt. % and of 5-Cb between 20 and 51 wt. %.

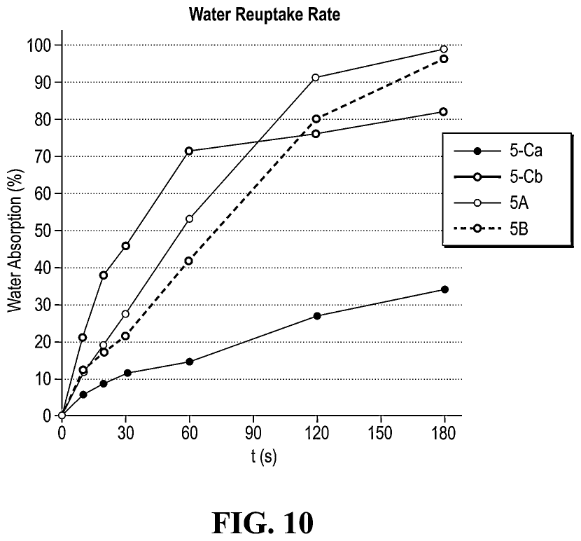

The plugs of Example 5 were tested for water reuptake by the procedure described in Example 4. The results of the water uptake rate for the media of Example 5 are shown in FIG. 10. As can be observed from FIG. 10, the plugs 5A, 5B rewetted regularly and fully within an acceptable time period. The plugs of comparative example 5-Cb rewetted irregularly as their moisture content varied wildly. The plugs of comparative example 5-Ca did not successfully rewet.

The media of Example 5 were also analyzed with regards to their porosity. The photographs of 12A-12D were taken under the same magnification next to a millimeter ruler for size reference. The photographs are of the external surface of the plugs. The distance of 1 cm is shown. FIG. 11A shows structure of the comparative example 5-Ca, FIG. 11B shows the comparative example 5-Cb, FIG. 11C shows Example 5A, and FIG. 11D shows Example 5B. As can be seen, porosity in the comparative examples is lower and differs from porosity of Examples 5A and 5B. Comparative example 5-Cb seems to have the lowest porosity. While comparative example 5-Ca features pores of various sizes, the porosity is less uniform than in Examples 5A and 5B. Examples 5A and 5B feature a greater number of pores relatively evenly or uniformly distributed through the body of the medium. Examples 5A and 5B also include pores of larger sizes than the comparative examples 5-Ca and 5-Cb.

### Example 6

Example 6 was set up to compare viability of the herein-disclosed medium for white spruce germination compared to traditional agar use. A tray of 28 plugs of the herein-disclosed medium containing both coir and peat fiber was prepared by a process described herein. The plugs were subsequently dried to the moisture content between about 9 and 15 wt. % and sterilized by gamma radiation set at about 35-50 kGy. The plugs of the tray were subsequently provided with mature somatic embryos of white spruce (Picea glauca). The photographs of FIGS. 12A and 12B taken were at the time of placement of 2-3 mm mature embryos on the herein-disclosed plug medium, and after a two-week period, respectively. After the two-week period, the embryos thrived and germinated as expected, and the plugs remained devoid of any unwanted life such as microorganisms, fungi, etc.

### Example 7

The trial of Example 7 was set up to investigate the rewettability of the herein-disclosed medium in comparison to competitor plugs with focus on rewettability. Table 3 below provides information about Example 7 and Comparative examples 7-Ca and 7-Cb.

TABLE 3
Properties of Example 7 and comparative examples 7-Ca and 7Cb (n = 8)

| | Moisture | Radiation dose | Average mass | Average mass |
|---|---|---|---|---|
| Example No. | content [wt. %] | [kGy] | [g, dry] | [g, wet] |
| 7 | 10.2 | 35 | 3.402 (±0.093) | 23.505 (±0.397) |
| 7-Ca | 8.5 | 35 | 2.460 (±0.064) | 10.729 (±0.266) |
| 7-Cb | 10.1 | 35 | 2.349 (±0.100) | 14.945 (±0.494) |

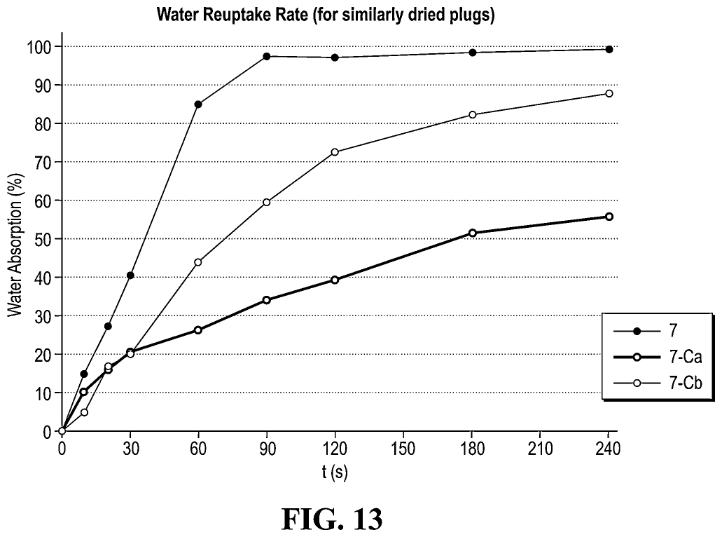

Two trays of each example and comparative examples were tested. All plugs had comparative fiber-portion composition (coir, peat mixture) and size (about 2.5 g, dry). All the plugs were dried in the same drying chamber and subsequently sterilized at similar radiation levels of 35 kGy. After sterilization, the plugs in trays were inserted in a packaging which was removed immediately before the test. Upon removal of the packaging, the plugs in the trays were subjected to a water uptake process described in Example 4. Results are shown in FIG. 13.

While plugs of Example 7 fully rewetted after 90 seconds, the comparative examples took up only about 25-60 wt. % of available moisture within the timeframe of 240 s. Generally, it was observed that comparative examples were much less consistently made, displaying relatively large differences in dried weight and mass at field capacity (when fully rewetted), thus jeopardizing predicable behavior for tissue culture process.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the disclosure that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A propagation plug comprising:
an irradiated, hydrophilic substrate body comprising a growing medium having bound organic fibers, the substrate body being rewettable and having
a moisture content of less than about 20 wt. %, based on the total weight of the body, as measured according to ASTM test method no. D2216 and
being sterile as evidenced by having an aerobic bacterial count of <10 cfu/g as measured by ISO test method no. 4833-1.

2. The propagation plug of claim 1, wherein the substrate body remains sterile for at least a month, as measured by ISO test method no. 4833-1.

3. The propagation plug of claim 1, wherein the organic fibers comprise peat and coir.

4. The propagation plug of claim 1, wherein the rewettable body retains its hydrophilicity and physical properties while repeatedly fluctuating between a state of low moisture content of as low as 6 wt. % and high moisture content of up to 96 wt. %, based on the weight of the substrate.

5. The propagation plug of claim 1, wherein the substrate body is generally cylindrical.

6. The propagation plug of claim 1, wherein the substrate body has a density of about 110-125 kg/m³.

7. The propagation plug of claim 1, wherein the substrate body has total porosity of about 90-95 vol. %, as measured by Eurofins test method no. FYS1: Cf PTOG nr 31 (1990).

8. The propagation plug of claim 1, wherein the substrate body has a top surface, a bottom surface, and a body surface extending between and connecting the top surface and the bottom surface, the top surface having a hole having a diameter of about 1-5 mm therein and a depth of about 3-15 mm.

9. The propagation plug of claim 1, wherein the substrate body is self-supporting.

10. The propagation plug of claim 1, wherein the substrate body includes antioxidants.

11. A method for forming a sterile propagation plug, the method comprising:
drying a substrate body comprising a growing medium having bound organic fibers to a moisture content of less than about 20 wt. %, as measured according to ASTM test method no. D2216; and
sterilizing the substrate body by exposure to radiation to generate a substantially sterile propagation plug as evidenced by having an aerobic bacterial count of <10 cfu/g, as measured by ISO test method no. 4833-1.

12. The method of claim 10, wherein the sterilizing comprises exposing the substrate body to about 20 to 90 kilograys.

13. The method of claim 10, wherein the sterilizing comprises providing a tray of 100 or more substrate bodies, the substrate body being one of the 100 or more substrate bodies, and exposing the tray of 100 or more substrate bodies to irradiation, the substrate body being disposed in the center of the tray and being confirmed to have a moisture content of below about 20 wt. %, based on the total weight of the substrate body.

14. The method of claim 13, wherein the tray of 100 or more substrate bodies includes bodies of substantially the same weight.

15. The method of claim 10, further comprising air-tight sealing the substrate body after drying and before sterilizing.

16. A tissue culture tray comprising:
a molded container including a plurality of cells;
a plurality of sterile, irradiated propagation plugs housed within the plurality of cells, each of the irradiated propagation plugs having a rewettable substrate body comprising a growing medium having bound organic fibers, the plurality of irradiated propagation plugs having substantially the same
dimensions,
moisture content of less than about 20 wt. %, based on the total weight of the body, as measured according to ASTM test method no. D2216, and
density of about 110-125 kg/m³, according to Eurofins method FYS1: Cf PTOG nr 31 (1990),
the plurality of sterile, irradiated propagation plugs being an initial tissue culture medium.

17. The tissue culture tray of claim 16, wherein the plurality of sterile, irradiated propagation plugs is hydrophilic.

18. The tissue culture tray of claim 16, wherein each one of the plurality of sterile, irradiated propagation plugs is sterile as evidenced by having an aerobic bacterial count of <10 cfu/g as measured by ISO test method no. 4833-1.

19. The tissue culture tray of claim 16, wherein each one of the plurality of sterile, irradiated propagation plugs retains its hydrophilicity and physical properties while repeatedly fluctuating between a state of low moisture content of as low as 6 wt. % and high moisture content of up to 100 wt. %, based on the weight of the substrate.

20. The tissue culture tray of claim 16, wherein each one of the plurality of sterile, irradiated propagation plugs has substantially the same weight.

21. The tissue culture tray of claim 16, further comprising a flowering plant initiated in each of the plurality of sterile, irradiated propagation plugs, the flowering plant having a system of ground roots.
